(19) United States
(12) Patent Application Publication
(10) Pub. No.: US 2025/0044259 A1
(43) Pub. Date: Feb. 6, 2025

(54) METHOD, SYSTEM AND SENSOR FOR DETERMINING PHYSICAL PROPERTIES OF CONCRETE AND OTHER MATERIALS

(71) Applicant: FILUMSENSE INC., Trumbull, CT (US)

(72) Inventor: Aleksander SCHILLER, Kiryat Biyalik (IL)

(21) Appl. No.: 18/716,277

(22) PCT Filed: Dec. 8, 2022
(86) PCT No.: PCT/IL2022/051300

§ 371 Date: Jun. 4, 2024

Related U.S. Application Data
(60) Provisional application No. 63/265,108, filed on Dec. 8, 2021.

(51) Int. Cl. G01N 29/12; G01N 29/24; G01N 29/46; G01N 33/38

(52) U.S. Cl. CPC G01N 29/12; G01N 29/2437; G01N 29/46; G01N 33/383; G01N 2291/0232; G01N 2291/102

(57) ABSTRACT

There is provided methods, systems and sensors for determining a compressive strength of concrete. A piezoelectric sensor is used to measure an ultrasound pulse response in the concrete and an ultrasound pulse frequency domain spectrum is calculated therefrom. A processor is used to determine, using a multivariable model, the compressive strength of the concrete using the ultrasound pulse frequency domain spectrum. The compressive strength of the concrete or an indication thereof is outputted.

### CROSS REFERENCE TO RELATED APPLICATIONS

The present disclosure claims priority to U.S. patent application No. 63/265,108 incorporated by reference herein.

### TECHNOLOGICAL FIELD

The present disclosure generally relates to methods, systems and sensors for determining compressive strength of concrete, and more particularly relates to methods, systems and sensors for determining compressive strength of concrete by implementing a multivariable model (or multivariate analysis) based on an ultrasound pulse frequency domain spectrum of the concrete.

### BACKGROUND

Concrete is a material that has been used in construction for hundreds of years. It is a composite material comprised of aggregate bonded together with a fluid cement that hardens over time. The hardening of concrete is commonly referred to as setting. The particular composition of concrete can be varied considerably to achieve particular physical requirements from the set concrete.

It is important to monitor the setting process of concrete so that the strength of the concrete can be verified for safety reasons and so that subsequent steps of construction (or use of the concrete) can be carried out efficiently once a threshold setting level has been reached.

The compressive strength of the concrete is one important parameter which can be used to identify the setting level of the concrete. Prior attempts at measuring the compressive strength of concrete are either heavily dependent on a specific concrete mix being predetermined/predefined, involve a destructive test of the concrete and/or require calibration which delays useful measurements. For example, typical sensors require the sensor to be matched to a specific concrete mix and to be embedded in the concrete. A typical calibration period of 28 days thereafter is required before representative measurements may be obtained from the embedded sensor.

Accordingly, it is desirable to provide universal, non-destructive methods, systems and sensors that are capable of accurate determination of physical properties of concrete, such as compressive strength, without the need for calibration. Furthermore, other desirable features and characteristics of the present disclosure will become apparent from the subsequent detailed description and the appended claims, taken in conjunction with the accompanying drawings and the foregoing technical field and background.

### GENERAL DESCRIPTION

In one aspect, a method of determining a compressive strength of concrete is provided. The method comprises: measuring, using a piezoelectric sensor, an ultrasound pulse response in the concrete and calculating an ultrasound pulse frequency domain spectrum therefrom; determining, using a processor and a multivariable model, the compressive strength of the concrete using the ultrasound pulse frequency domain spectrum; and outputting the compressive strength of the concrete or an indication thereof (i.e. outputting data indicative of the compressive strength of the concrete).

In embodiments, at least two metrics from the ultrasound pulse response and/or the ultrasound pulse frequency domain spectrum are used in the multivariable modelling.

In embodiments, the at least two metrics include two or more of: an ultrasound pulse velocity, a harmonic onset time, a peak frequency, a peak amplitude, a peak area, a peak slope and a peak-to-peak ratio.

In embodiments, the method further comprises: measuring an electromechanical impedance response in the concrete and calculating an electromechanical impedance frequency domain spectrum therefrom, wherein determining, using the processor and the multivariable model, the compressive strength of the concrete comprises using the ultrasound pulse frequency domain spectrum and the electromechanical impedance frequency domain spectrum.

In embodiments, the piezoelectric sensor is used to measure the electromechanical impedance frequency response in the concrete.

In embodiments, at least one metric from the electromechanical impedance frequency domain spectrum is used in the multivariable modelling.

In embodiments, the at least one metric includes one or more of: a peak frequency, a peak amplitude, a peak area, a peak slope and a peak-to-peak ratio.

In embodiments, the method further comprises measuring, using a temperature sensor, a temperature in the concrete.

In embodiments, the temperature is used in the multivariable modelling as a correction factor.

In embodiments, the piezoelectric sensor is at least partially embedded in the concrete and/or wherein the piezoelectric sensor is disposed on an external surface of the concrete. Additionally/alternatively, the piezoelectric sensor is disposed on an external surface of the concrete such that a piezoelectric transducer of the piezoelectric sensor is disposed outside of the concrete.

In embodiments, the output of the compressive strength of the concrete is used to determine an amount by which the concrete has set.

In embodiments, determining the compressive strength of the concrete using the multivariable model comprises using a machine learning model, such as a neural network model or a decision-tree based model.

In embodiments, the neural network model has been trained using a plurality of concrete samples with known properties.

In embodiments, the known properties include at least one of: the compressive strength, the specified compressive strength, the ultrasound pulse velocity frequency domain spectrum and the electromechanical impedance frequency domain spectrum.

In embodiments, the concrete samples with known properties are randomly chosen from different ready mix concrete suppliers at different periods of time. In certain embodiments, a fully independent validation set is randomly chosen thereafter.

In embodiments, the method further comprises measuring the humidity of the concrete and determining the compressive strength of the concrete additionally using the measured humidity.

In embodiments, the measured humidity is used to correct the ultrasound pulse velocity frequency domain spectrum and/or the electromechanical impedance frequency domain spectrum.

In embodiments, the method further comprises measuring the electrical conductivity of the concrete and determining the compressive strength of the concrete additionally using the measured electrical conductivity.

In embodiments, the method further comprises measuring the pH of the concrete and determining the compressive strength of the concrete additionally using the measured pH.

In embodiments, the method further comprises determining the compressive strength of the concrete additionally using at least one of: the specified concrete density, the specified air content and the specified slump.

In embodiments, the specified concrete density, the specified air content and the specified slump may be the delivery ticket values provided by the manufacturer of the concrete mix.

In another aspect, a method of determining a compressive strength of concrete is provided. The method comprises: measuring, using a piezoelectric sensor, an ultrasound pulse response in the concrete and calculating, using a processor, an ultrasound pulse frequency domain spectrum therefrom; measuring, using the piezoelectric sensor, an electromechanical impedance response in the concrete and calculating, using the processor, an electromechanical impedance frequency domain spectrum therefrom; measuring, using a temperature sensor, a temperature of the concrete; repeating the measurements of the ultrasound pulse response, the electromechanical impedance response and the temperature over time as the concrete sets, and calculating, using the processor, corresponding ultrasound pulse frequency domain spectrums and electromechanical impedance frequency domain spectrums therefrom; and determining, using the processor and a multivariable model, the compressive strength of the concrete using the multiple ultrasound pulse frequency domain spectrums and the multiple electromechanical impedance frequency domain spectrums, wherein the multivariable modelling includes using the temperature as a correction factor; and outputting a compressive strength of the concrete or an indication therefore.

In embodiments, a derivative of an ultrasound pulse velocity with respect to temperature and/or a derivative of the ultrasound pulse frequency domain spectrum with respect to temperature and/or a derivative of the electromechanical impedance frequency domain spectrum with respect to temperature is used as the correction factor.

In a further aspect, there is provided a sensor for determining the compressive strength of concrete. The sensor comprises: a piezoelectric transmitter; and a piezoelectric receiver, wherein the piezoelectric transmitter and the piezoelectric receiver are configured to act together to measure an ultrasound pulse response in the concrete and an electromechanical impedance response in the concrete.

In embodiments, the piezoelectric transmitter and the piezoelectric receiver are spaced apart to define a gap, and wherein the gap is configured to receive a portion of the concrete.

In embodiments, the piezoelectric transmitter and the piezoelectric receiver are implemented in a piezoelectric transducer acting both as the piezoelectric transmitter and receiver.

In embodiments, the sensor is configured to measure the ultrasound pulse response and the electromechanical impedance response with a dwell time therebetween. Optionally, the dwell time is of a length to prevent cross talk between the measured ultrasound pulse response and the measured electromechanical impedance response.

In embodiments, the sensor is configured to measure the ultrasound pulse response and the electromechanical impedance response with a dwell time therebetween, optionally, wherein the dwell time is between approximately 1 and approximately 10 seconds, approximately 3 and approximately 8 seconds, or approximately 5 seconds.

In embodiments, the sensor is configured to perform multiple measurements of both the ultrasound pulse response and the electromechanical impedance response, and wherein the multiple measurements are at time intervals.

In embodiments, the sensor further comprises a temperature sensor configured to measure a temperature of the concrete.

In embodiments, the piezoelectric transmitter and/or the piezoelectric receiver comprises an acoustic coupling layer configured to come into contact with the concrete.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a block diagram of a system for determining a compressive strength of concrete;

FIG. 2 is a schematic representation of a sensor of the system shown in FIG. 1;

FIG. 3 shows a schematic representation of a piezoelectric transmitter/receiver of the sensor of FIG. 2.

FIGS. 4A-4C show different arrangements for the sensor shown in FIG. 2;

FIG. 5 is a flow chart showing a method of determining a compressive strength of concrete;

FIG. 6 is a flow chart showing details of the manner in which the compressive strength is calculated;

FIGS. 7A-7B show an exemplary ultrasound pulse response and a corresponding ultrasound pulse frequency domain spectrum, respectively;

FIGS. 8A-8B show how the ultrasound pulse frequency domain spectrum and the electromechanical impedance frequency domain spectrum, respectively, evolve over time as concrete dries; and

FIG. 9 shows an alternative piezoelectric sensor.

### DETAILED DESCRIPTION OF EMBODIMENTS

As used herein, a specified physical parameter of concrete, such as the specified compressive strength, refers to a predetermined/pre-measured value of this physical parameter, as would be understood by those skilled in the art. Concrete manufacturers measure these specified physical parameters and advertise them with the different mixes of concrete, for example on the mix ticket (certificate of conformance). For example, a specified compressive strength may be advertised as B30 which means that the compressive strength of the concrete will be at least 30 MPa after 28 days from pouring.

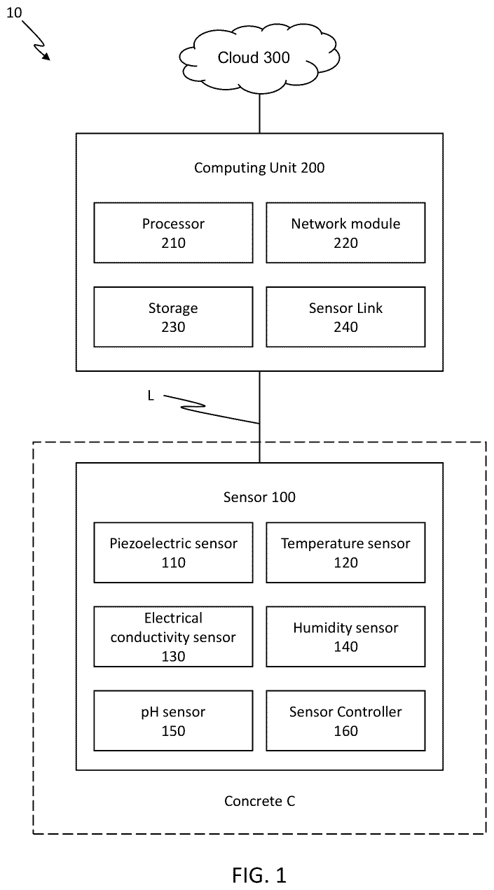

FIG. 1 is a block diagram of a system 10 for determining a compressive strength of concrete C. The system 10 generally comprises at least one sensor 100, at least one computing unit 200 and at least one cloud 300 (e.g. a remote server). The sensor 100 is arranged to communicate with the computing unit 200 which in turn may communicate with the cloud 300.

The sensor 100 is configured to be arranged entirely or partially within the concrete C, and/or on an external surface thereof. The sensor 100 is configured to take various measurements of physical properties of the concrete C over time as the concrete C sets. As described below, the sensor 100 may take sets of measurements repeatedly as the concrete C sets, for example, sets of measurements may be repeated daily to monitor the setting process of the concrete C.

The sensor 100 comprises a number of sensor elements configured to measure various physical properties of the concrete C at particular times during the setting process of the concrete C. The measurements from these sensor elements may be used to determine other properties of the concrete C, such as the compressive strength, as it evolves during the setting process.

The sensor elements of the sensor 100 include a piezoelectric sensor 110, a temperature sensor 120, an electrical conductivity sensor 130, a humidity sensor 140 and a pH sensor 150 each configured to measure physical properties (described below) of the concrete C. Those skilled in the art would understand that further sensors may be included in sensor 100 and/or some of the above sensor elements may be excluded.

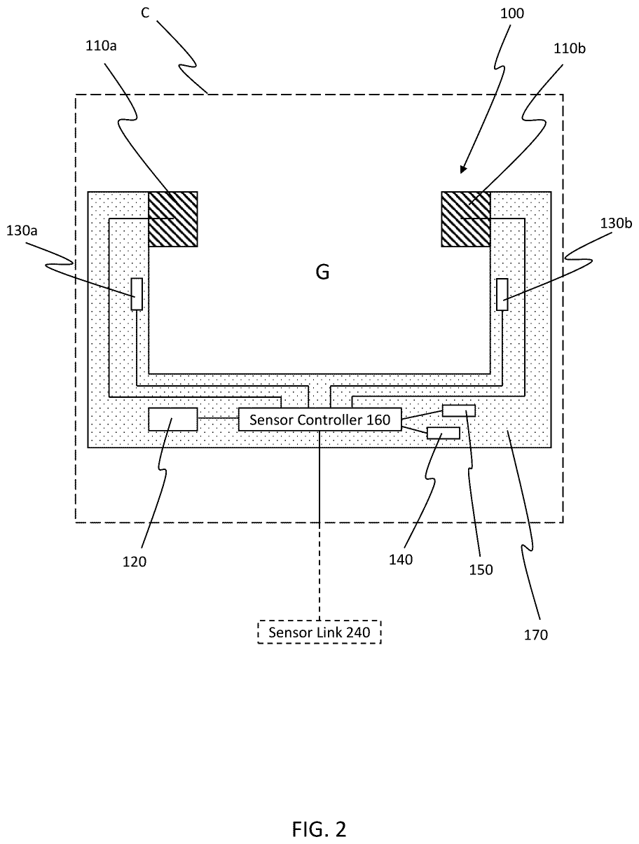

The sensor 100 also comprises sensor controller 160. The sensor controller 160 may be inside the concrete C (as shown in FIGS. 1 and 2) or alternatively may be located outside of the concrete C. Each of the piezoelectric sensor 110, the temperature sensor 120, the electrical conductivity sensor 130, the humidity sensor 140 and the pH sensor 150 is connected to the sensor controller 160 such that each sensor element can relay their respective measured data to the sensor controller 160. The sensor controller 160 also acts to power and control each of the piezoelectric sensor 110, the temperature sensor 120, the electrical conductivity sensor 130, the humidity sensor 140 and the pH sensor 150. For example, the sensor controller 160 can selectively control each sensor element so that it takes measurements of their respective parameters on command from the sensor controller 160.

The sensor controller 160 is also configured to communicate with corresponding sensor link 240 of the computing unit 200 via a link L. Any method of communication is suitable, for example, by a wired connection or a wireless connection, of which various alternatives would be known to those skilled in the art. If the link L between sensor controller 160 of the sensor 100 and the sensor link 240 of computing unit 200 is wired, the link L may be configured to provide power to the sensor 100 suitable for operation of the sensor 100. If the link L between sensor controller 160 of the sensor 100 and the sensor link 240 of computing unit 200 is wireless, the sensor 100 may comprise a battery (not shown) which may be configured to provide power to the sensor 100 suitable for operation of the sensor 100.

The sensor 100 may be configured to be retrievable once all required measurements of the concrete C have been made. For example, the sensor 100 may be retrieved once the concrete C has reached a suitable set level (e.g. after 28 days). The sensor 100 may be retrievable by breaking a section of the set concrete C. Additionally/alternatively, the sensor 100 may be configured to be disposable such that no retrieval is required and the sensor 100 may be left in the concrete C even during use of the concrete C. For example, the sensor 100 may remain in the concrete C for Structural Health Monitoring (SHM) purposes.

The computing unit 200 comprises a processor 210, a network module 220, storage 230 and sensor link 240. The computing unit 200 may be disposed anywhere in relation to the sensor 100, so long as the link L is able to allow for communication between the sensor controller 160 of the sensor 100 and the sensor link 240 of the computing unit 200. For example, the computing unit 200 may be located relatively close to the sensor 100 (for example next/adjacent to the concrete C) or located remotely in a centralized location on a construction site where the concrete C is located. Additionally/alternatively, the computing unit 200 may not be separate from the sensor 100 (as shown in FIG. 1) and instead some or all components of the computing unit 200 may be integral with the sensor 100. For example, the processor 210, the network module 220 and the storage 230 may be integral with the sensor 100. Specifically, the sensor controller 160, the processor 210, the network module 220 and the storage 230 may be provided as a system-on-chip (SoC) within the sensor 100.

The computing unit 200 is configured to receive data from sensor 100 via the link L. The data includes the measured data from the sensor elements, such as the piezoelectric sensor 110, the temperature sensor 120, the electrical conductivity sensor 130, the humidity sensor 140 and the pH sensor 150. The data received from the sensor 100 may be the raw data from some/all of the sensor elements and/or may be processed (to varying degrees) data from the sensor elements.

The computing unit 200 is configured to process the data received from the sensor 100 and provide outputs relating to the state of the concrete C, for example, the compressive strength of the concrete C. The results from the computing unit 200 may enable a determination of the level of setting of the concrete C.

The sensor link 240 is configured to receive the data from the sensor controller 160 of the sensor 100 via link L. The received data is then processed (see below) by the processor 210, for example, to determine the compressive strength of the concrete C. Optionally, the storage 230 may include more than one type of storage device. The storage 230 be used during the processing of the data by the processor 210 and/or as a more permanent form of storage before/after the processing is completed by the processor 210. The storage 230 may comprise any combination of volatile memory and non-volatile memory, as would be known to those skilled in the art. For example, the storage 230 may be implemented using any of a number of known storage/memory devices such as PROMs (programmable read-only memory), EPROMS (electrically PROM), EEPROMs (electrically erasable PROM), flash memory, or any other electric, magnetic, optical, or combination storage/memory devices capable of storing data, some of which represent executable instructions, used by the processor 210.

Results from the processor 210, such as the determined compressive strength of the concrete C, may be stored on storage 230 and/or transmitted to the cloud 300 via network module 220 of the computing unit 200. The network module 220 may connect the computing unit 200 to the cloud via the internet. The cloud 300 may store and process the data further if required.

FIG. 2 is a schematic representation of the sensor 100 of the system 10 shown in FIG. 1. FIG. 2 is shown with the entire sensor 100 embedded in the concrete C. However, the sensor 100 may be arranged such that only a portion of the sensor 100 is within concrete C or such that the sensor is at least partially placed on an external surface of the concrete C.

The sensor 100 comprises various sensor elements, including the piezoelectric sensor 110 (shown in FIG. 2 as a piezoelectric transmitter 110a and a piezoelectric receiver 110b), the temperature sensor 120, the electrical conductivity sensor 130, the humidity sensor 140, the pH sensor 150. The sensor 100 also comprises the sensor controller 160 which is in communication with the sensor link 240 of the computing unit 200.

The sensor 100 comprises a body 170 which is configured to secure each of the piezoelectric sensor 110a, 110b, the temperature sensor 120, the electrical conductivity sensor 130, the humidity sensor 140, the pH sensor 150 and the sensor controller 160. The body 170 is rigid such that negligible flexing of the body 170 occurs during use of the sensor 100. This allows the various sensors to stay in a fixed position relative to each other, which may help to obtain consistent and accurate data. For example, the body 170 is rigid such that the distance between the piezoelectric transmitter 110a and the piezoelectric receiver 110b is generally fixed.

The piezoelectric sensor 110a, 110b, the temperature sensor 120, the electrical conductivity sensor 130, the humidity sensor 140, the pH sensor 150 are each in communication with the sensor controller 160. The communication can be direct electrical communication via electrical wires between the sensor elements and the sensor controller 160. Additionally/alternatively, some/all of the sensor elements may be in wireless communication with the sensor controller 160. In such configurations, each sensor element may have an individual controller and power supply (such as a cell/battery). The sensor controller 160 acts to control each of the piezoelectric sensor 110a, 110b, the temperature sensor 120, the electrical conductivity sensor 130, the humidity sensor 140, the pH sensor 150 such that they take measurements of their respective physical parameters of the concrete C. As described above, the sensor controller 160 also communicates with the sensor link 240 of the computing unit 200, for example, to send the measurement data to the computing unit 200.

The sensor 100 defines a gap G for receiving a portion of the concrete C. The concrete C in the gap G is in the proximity of the piezoelectric sensor 110a, 110b, the temperature sensor 120, the electrical conductivity sensor 130, the humidity sensor 140 and the pH sensor 150. In use, the sensor 100 measures physical properties of the concrete C in the gap G, as described below.

The gap G may be of any suitable size known to those skilled in the art. For example, the gap G may define a total volume of approximately 5000 cm3 to approximately 10,000 cm3, optionally, approximately 6000 cm3 to approximately 9000 cm3, and further optionally, approximately 7000 cm3 to approximately 8000 cm3.

The distance between the piezoelectric transmitter 110a and the piezoelectric receiver 110b may be between approximately 1.5 cm to 1.5 m. Optionally, the distance between the piezoelectric transmitter 110a and the piezoelectric receiver 110b may be between approximately 5.5 cm to 15 cm. This specific range is particularly useful in concrete C as it is sufficiently greater than the nominal aggregate size in the concrete C (providing sufficient averaging) but balances the energy management of the piezoelectric sensor 110 as it does not require a much higher voltage as would be needed for larger distances.

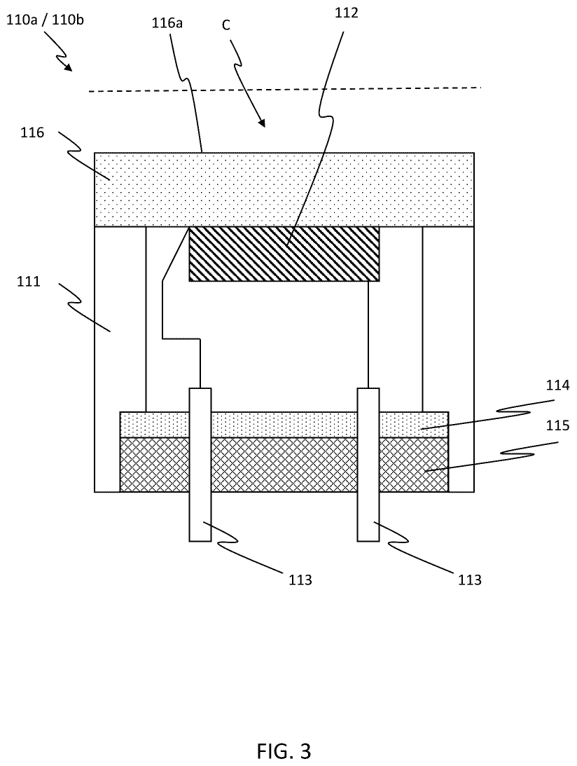

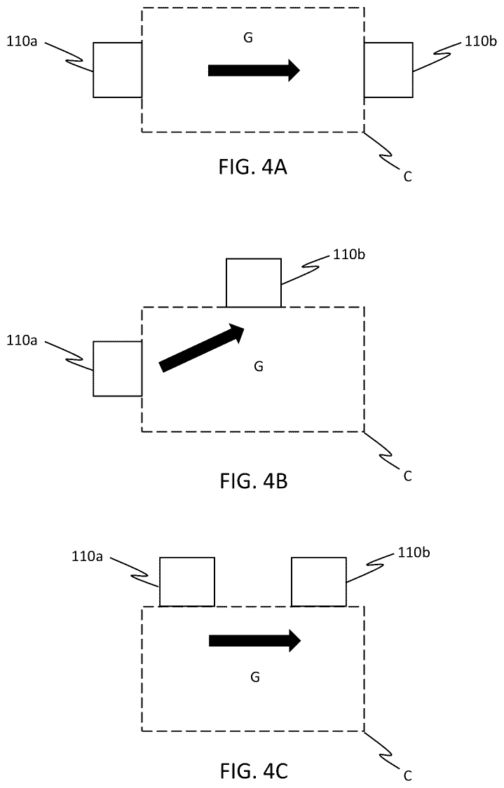

The piezoelectric sensor 110 can be used to measure the ultrasound response in the concrete C and/or the electromechanical impedance (EMI) response in the concrete C. The piezoelectric sensor 110 comprises at least two elements, namely, a piezoelectric transmitter 110a and a piezoelectric receiver 110b (see FIGS. 4A to 4C for further details on their potential relative arrangements within the sensor 100). Both of the piezoelectric transmitter 110a and the piezoelectric receiver 110b comprise at least one piezoelectric transducer. Further details of the piezoelectric transmitter 110a and the piezoelectric receiver 110b are descried below in relation to FIG. 3. The sensor controller 160 controls and/or monitors each of the piezoelectric transducers in the piezoelectric transmitter 110a and the piezoelectric receiver 110b, in a manner which would be familiar to those skilled in the art.

The sensor 100 is able to measure the ultrasound response in the concrete C. Specifically, to measure the ultrasound response, firstly, the sensor controller 160 provides a pulse of alternating current across the piezoelectric transducer of the piezoelectric transmitter 110a, thereby causing the piezoelectric transducer of the piezoelectric transmitter 110a to vibrate, as would be understood by those skilled in the art. The vibrations in the piezoelectric transducer of the piezoelectric transmitter 110a are transmitted to the concrete C and propagate therethrough.

These vibrations propagate through concrete C in the gap G and impart corresponding vibrations on the piezoelectric transducer of the piezoelectric receiver 110b. The piezoelectric transducer of the piezoelectric receiver 110b vibrates correspondingly which creates a corresponding voltage across the piezoelectric transducer of the piezoelectric receiver 110b. This voltage can be measured by the sensor controller 160. Accordingly, by controlling the electrical signal provided to the piezoelectric transducer of the piezoelectric transmitter 110a (and the duration and frequency thereof), the sensor controller 160 can send an ultrasonic pulse through the concrete C and measure an ultrasonic pulse response by measuring the voltage across the piezoelectric transducer of the piezoelectric receiver 110b.

As detailed below, the ultrasonic pulse response measured by the piezoelectric sensor 110 contains information regarding the physical properties of the concrete C due to the manner in which the ultrasound pulse is attenuated and/or modulated as it propagates through the material of the concrete C.

The same piezoelectric sensor 110 can be controlled by the sensor controller 160 to additionally measure the electromechanical impedance response in the concrete C. Specifically, using the piezoelectric receiver 110b (and/or the piezoelectric transmitter 110a), the sensor controller 160 may measure the electrical impedance of the piezoelectric transducer of the piezoelectric receiver 110b. As the physical properties of the concrete C (such as the stiffness, compressive strength, shrinkage, etc.) impart a strain on the piezoelectric transducer of the piezoelectric receiver 110b, the electrical impedance of the piezoelectric transducer of the piezoelectric receiver 110b can be used to determine physical properties of the concrete C.

The sensor 110 further comprises the temperature sensor 120. The temperature sensor 120 may be any temperature sensor that is capable of measuring the temperature of the concrete. The temperature sensor 120 may be suitably located such that other parts of the sensor 100 (e.g. heat generating parts) do not influence the temperature readings of the temperature sensor 120. To this end, the temperature sensor 120 may be disposed adjacent to the concrete C, and optionally, in contact with the concrete C. The temperature sensor 120 may be a thermocouple/thermoelectrical thermometer and/or a thermal camera.

The sensor 100 further comprises the electrical conductivity sensor 130. The electrical conductivity sensor 130 is configured to measure the electrical conductivity of the concrete C in the gap G. The electrical conductivity sensor 130 comprises a first electrode 130a and a second electrode 130b. The first electrode 130a and the second electrode 130b may be distinct elements or parts of the body 170 (e.g. arms of the body 170 which hold other components). To measure the electrical conductivity in the concrete C, the sensor controller 160 applies a current between the first electrode 130a and the second electrode 130b which flows through the concrete C. The sensor controller 160 is configured to determine the electrical conductivity based on the applied current, resistance and physical geometrical properties of the concrete C, as would be known to those skilled in the art.

The sensor 100 additionally comprises a humidity sensor 140. The humidity sensor 140 is configured to measure the humidity of the concrete C in the gap G. Any suitable humidity sensor known to those in the art may be used in sensor 100, so long as the humidity of the concrete C may be measured. The humidity sensor 140 may measure the absolute humidity and/or the relative humidity.

The sensor 100 additionally comprises a pH sensor 140. The pH sensor 140 is configured to measure the pH of the concrete C in the gap G. Any suitable pH sensor known to those in the art may be used in sensor 100, so long as the pH of the concrete C may be measured.

The sensor 100 additionally may optionally comprise a rebar corrosion indicator (not shown).

FIG. 3 shows a schematic representation of a piezoelectric transmitter 110a or a piezoelectric receiver 110b of the sensor 100 of FIG. 2. The illustration provided in FIG. 3 applies to both of the piezoelectric transmitter 110a and the piezoelectric receiver 110b of the sensor 100. FIG. 3 shows the piezoelectric transmitter 110a/the piezoelectric receiver 110b in contact with concrete C.

The piezoelectric transmitter 110a and the piezoelectric receiver 110b each may comprise a frame 111, a piezoelectric transducer 112, two or more electrodes 113, a base 114, a shielding piece 115 and an acoustic coupling layer 116 defining an ultrasonic radiation surface 116a.

The frame 111 and base 114 generally provide the structural backbone of the piezoelectric transmitter 110a/piezoelectric receiver 110b and are rigid such that the various other elements are held securely in place, even during operation.

The frame 111 and base 114 (and optionally other components of the sensor 100, especially those that come into direct contact with the concrete C) may be hermetically sealed. This allows the sensor to operate in a very aggressive concrete environment with a starting pH of approximately 13.

In the configuration of the piezoelectric transmitter 110a, the piezoelectric transducer 112 is driven by application of a voltage across the electrodes 113 by sensor controller 160. As shown in FIG. 3, the voltage is applied across the width of the piezoelectric transducer 112 which causes mechanical strain in the piezoelectric transducer 112, as would be understood by those skilled in the art. Depending on the application of voltage by the sensor controller 160, the piezoelectric transducer 112 can be made to vibrate.

In the configuration of the piezoelectric receiver 110b, any mechanical strain applied to the piezoelectric transducer 112 (e.g. from vibrations acting on the piezoelectric transducer 112) causes a corresponding voltage to form across the piezoelectric transducer 112. This voltage can be measured across the electrodes 113 by the sensor controller 160. Depending on the particular form of mechanical strain applied on the piezoelectric transducer 112, the voltage measured by the sensor controller 160 will vary.

The acoustic coupling layer 116 is placed in contact with a surface of the piezoelectric transducer 112. For example, as shown in FIG. 3, an entire surface of the piezoelectric transducer 112 is flush with a portion of the acoustic coupling layer 116. The acoustic coupling layer 116 defines an ultrasonic radiation surface 116a. The acoustic coupling layer 116 assists in transferring energy from the piezoelectric transducer 112 to the concrete C that is in contact with the ultrasonic radiation surface 116a.

The shielding piece 115 acts to prevent/minimize energy from the piezoelectric transducer 112 being transmitted therethrough, for example, into the rest of the sensor 100. This minimizes cross-talk between sensor elements in the sensor 100 and ensures that measurements made by the piezoelectric transducer 112 are sensitive to the concrete C only rather than other elements.

FIGS. 4A to 4C show different geometric arrangements of the piezoelectric transmitter 110a and the piezoelectric receiver 110b.

FIG. 4A shows an arrangement where the piezoelectric transmitter 110a and the piezoelectric receiver 110b are generally opposite to each other (e.g., oppositely across the gap G). In such configurations, the respective ultrasonic radiation surfaces 116a (see FIG. 3) of the piezoelectric transmitter 110a and the piezoelectric receiver 110b generally face each other (e.g., at opposing sides of the gap G). Optionally, the piezoelectric transducer of the piezoelectric transmitter 110a is generally parallel to the piezoelectric transducer of the piezoelectric receiver 110b. This arrangement may be used to measure the direct ultrasound pulse response.

FIG. 4B shows an arrangement where the piezoelectric transmitter 110a and the piezoelectric receiver 110b are angled to each other (e.g., diagonally across the gap G). In such configurations, the respective ultrasonic radiation surfaces 116a (see FIG. 3) of the piezoelectric transmitter 110a and the piezoelectric receiver 110b are generally angled/perpendicular with respect to each other (e.g., at adjacent sides of the gap G). Optionally, the piezoelectric transmitter 110a and the piezoelectric receiver 110b may be generally perpendicular to each other. Optionally, the piezoelectric transducer of the piezoelectric transmitter 110a may be angled/generally perpendicular to the piezoelectric transducer of the piezoelectric receiver 110b. This arrangement may be used to measure the semi-direct ultrasound pulse response.

FIG. 4C shows an arrangement where the piezoelectric transmitter 110a and the piezoelectric receiver 110b are generally next to each other, for example, on the same surface of the concrete C (i.e., on the same side of the gap G). In such configurations, the respective ultrasonic radiation surfaces 116a (see FIG. 3) of the piezoelectric transmitter 110a and the piezoelectric receiver 110b are generally in the same plane to each other (e.g., on the same side of the gap G). Optionally, the piezoelectric transducer of the piezoelectric transmitter 110a is generally parallel to the piezoelectric transducer of the piezoelectric receiver 110b. This arrangement may be used to measure the indirect and/or surface ultrasound pulse response.

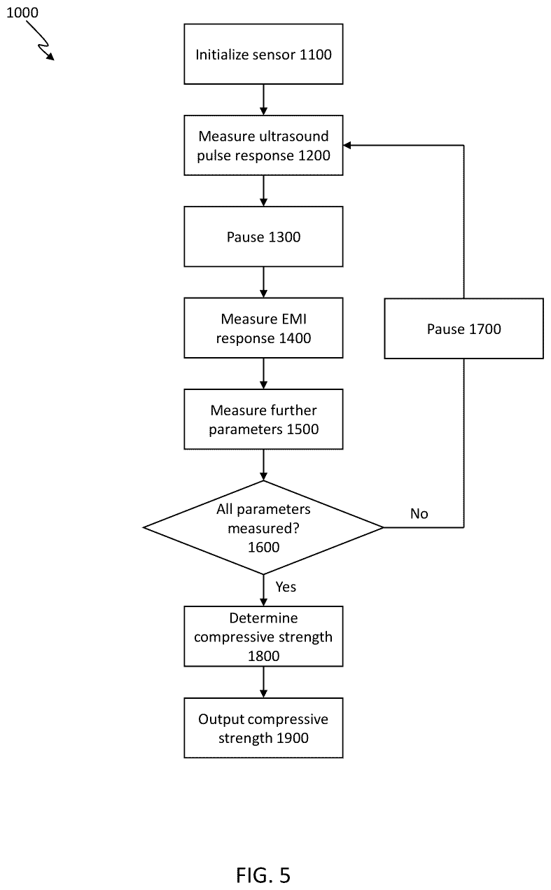

FIG. 5 is a flow chart showing a method 1000 of determining a compressive strength of concrete C. The steps of the flow chart of FIG. 5 can be implemented by computer program instructions running on the computing unit 200 and/or the sensor controller 160. The computer program instructions may be stored on the sensor controller 160, storage 230 of the computing unit 200 and/or the cloud 300.

The method 1000 of determining a compressive strength of concrete C starts at step 1100 with the initialization of the sensor 100. This may occur in response to a start instruction from the computing unit 200. For example, the computing unit 200 may communicate with the sensor controller 160 to send a start signal to the sensor 100. The start signal may cause the sensor 100 to initialize, for example, by powering on the various sensor elements such that they are ready to take measurements.

The method 1000 repeatedly carries out sets of measurements of the properties of the concrete C at specific time intervals, starting at an initial time (time=0 days). At step 1200, a measurement of the ultrasound pulse response of the concrete C is performed using the piezoelectric sensor 110 as described above. The measurement of the ultrasound pulse response may be processed immediately or processed once other parameters are measured.

At step 1300, the method 1000 pauses for a period of time, namely, the dwell time. During this period, no measurements are conducted on the concrete C. This reduces the chances of cross-talk between the various measurements which assists in providing consistent and accurate results. The pause 1300 is most critical between the measurement of the ultrasound pulse response at step 1200 and the measurement of the electromechanical impedance response at step 1400 (described below). For brevity, the pause 1300 is shown only between measurement step 1200 and measurement step 1400, however, a pause may be included between all measurements the sensor 100 takes.

The dwell time in pause 1300 between the measurement of the ultrasound pulse response at step 1200 and the measurement of the electromechanical impedance response at step 1400 may be between approximately 1 and approximately 10 seconds, optionally, approximately 3 and approximately 8 seconds, and further optionally, approximately 5 seconds.

In embodiments, the dwell time is of a length to prevent cross talk between the measured ultrasound pulse response and the measured electromechanical impedance response.

The pause 1300 (and any other pause steps) are entirely optional. For example, in embodiments where the electromechanical impedance response is measured using a separate sensor than that used to measure the ultrasound pulse response (see below), a pause may not be required.

At step 1400, a measurement of the electromechanical impedance response of the concrete C is performed using the piezoelectric sensor 110 as described above. The measurement of the electromechanical impedance response may be processed immediately or processed once other parameters are measured.

At step 1500, any further measurements of parameters is carried out by the sensor 100, with or without a pause between measurements. For example, the sensor 100 may then measure any combination of: the temperature using temperature sensor 120, the electrical conductivity using the electrical conductivity sensor 130, the humidity using the humidity sensor 140, and the pH using the pH sensor 150. Again, the measurements of these various parameters may be processed immediately or processed once other parameters are measured.

The above-noted various measurements carried out at an initial time (e.g. time=0 days). Accordingly, the various measurements (a set of measurements) are associated with the state of the concrete C at a particular time (e.g. time=0 days). Therefore, the above-noted set of measurements may optionally be carried out within a short time period of each other, for example, all measurements in the set of measurements may be carried out within a total time of 1 minute or less.

To monitor how the concrete C sets over time (typically over a period of many days), the sensor 100 will repeat the above steps at various time intervals to form several sets of measurements over time. For example, at step 1600, the sensor may repeat the above-noted measurements in steps 1200, 1300, 1400, 1500 at various time intervals, e.g. daily, to form several sets of measurements which are representative of the state of the concrete C over time. The pause 1700 may have a duration of an hour, 6 hours, 12 hours, or 24 hours (optionally, minus the time taken for the various measurements to be conducted). The pause 1700 may be configured to be optionally changed throughout the operation of the sensor 100 (for example, by customer demand).

Step 1600 checks whether all sets of measurements have been obtained. This may be checked against a predetermined number (e.g. 28 set of measurements to represent 28 days of setting), or may be dynamically updated by the computing unit 200 and/or the cloud 300, e.g. by the sending of a stop command when sufficient readings have been taken. This update may be provided to the sensor 100 via the link L.

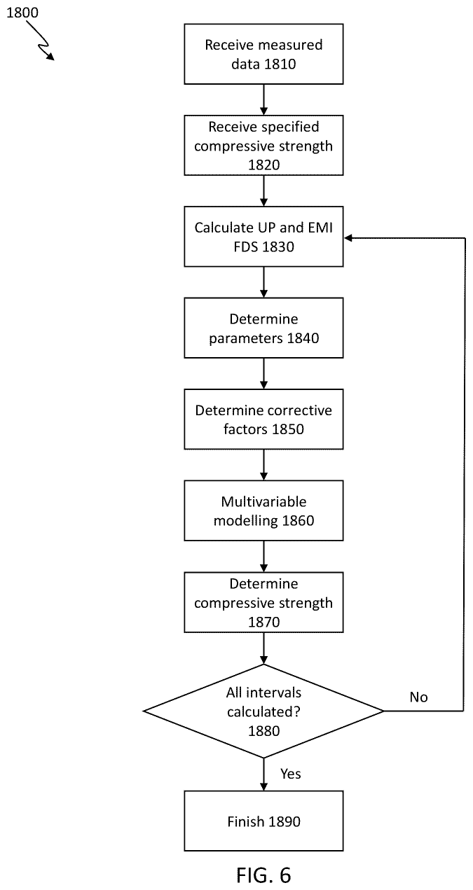

At step 1800, the results of the measurements are processed to determine the compressive strength of the concrete C at the various time intervals (e.g. daily). The processing of the measurements may be carried out by the computing unit 200, in particular, the processor 210 of the computing unit 200 (edge approach). Additionally/alternatively, some/all of the processing may be carried out by the sensor 100, e.g. by the sensor controller 160 (edge approach). The processing of the measurements may occur in real time (e.g. after each measurement is taken) or once all measurements are conducted. The specifics of determining the compressive strength of the concrete C are described in relation to FIG. 6 below. In certain embodiments, all processing of the measurements may be carried out by the cloud 300 (cloud approach).

At step 1900, the compressive strength of the concrete C is outputted. For example, the computing unit 200 may display the determined compressive strengths of the concrete C on a user interface (not shown) and/or upload the results to the cloud 300 for users to access remotely. The determined compressive strength may subsequently used to determine how much the concrete C has set by. Additionally/alternatively, at step 1900, an indication of the compressive strength of the concrete C is outputted, for example, as a percent of the specified compressive strength.

FIG. 6 is a flow chart showing details of the manner in which the compressive strength is calculated based on the sets of measurements obtained in steps 1100 to 1600 shown in FIG. 5. Some/all of the steps shown in FIG. 6 may be carried out as soon as a particular measurement is completed, after each complete set of measurements (at a specific time, e.g. time=0 days) is completed, and/or after all sets of measurements is complete. In many circumstances, it is useful to perform the calculations as soon as a complete set of measurements for a specified time (e.g. time=0 days) is available.

At step 1810, the measured data from any/all of the previous steps 1200, 1300, 1400, 1500 is received. This may involve the processor 210 and/or the storage 230 receiving the data. At step 1820, a specified compressive strength of the concrete C may optionally be received. Step 1820 may occur at any time, in particular, the step 1820 may be carried out before step 1110. The specified compressive strength of the concrete C may be entered by the user on a user interface of the computing unit 200 or entered using any other computing device connected to the cloud 300 so as to be supplied to the computing unit 200. As understood by those skilled in the art, the specified compressive strength of the concrete C may be a predetermined/pre-measured compressive strength of the concrete C at a particular time, for example, after 28 days of pouring. The specified compressive strength of the concrete C may be obtained from the manufacturer of the concrete mix and is typically provided on the delivery ticket of the concrete mix. Additionally/alternatively, the specified concrete may have been previously measured using any technique known to those skilled in the art, including any destructive technique of a sample of the concrete C. Step 1820 is purely optional and a specified compressive strength may not be used in method 1800 at all.

At step 1830, the ultrasound pulse response data is Fourier Transformed (FT)/Fast Fourier Transformed (FFT)/wavelet transformed to obtain an ultrasound pulse frequency domain spectrum.

At step 1840, various parameters may be determined for use in the subsequent multivariable modelling (see step 1860). Specifically, the ultrasound pulse response and the calculated ultrasound pulse frequency domain spectrum from step 1830 may be used to determine any combination of: an ultrasound pulse velocity, a harmonic onset time, a peak frequency, a peak amplitude, a peak area, a peak slope and a peak-to-peak ratio of the ultrasound pulse response. Additionally, the electromechanical impedance response and the calculated electromechanical impedance frequency domain spectrum from step 1830 may be used to determine any combination of: a peak frequency, a peak amplitude, a peak area, a peak slope and a peak-to-peak ratio of the electromechanical impedance response.

Optionally, at step 1850, any corrective factors are calculated. For example, at step 1850, the temperature measurements from the temperature sensor 120 may be used to determine one or more corrective factors to be used in the subsequent multivariable modelling 1860. Specifically, a derivative of an ultrasound pulse velocity with respect to temperature, namely, d(UPV)/dT), and/or a derivative of the ultrasound pulse frequency domain spectrum with respect to temperature, namely, d(UP FDS)/d(T), and/or a derivative of the electromechanical impedance frequency domain spectrum with respect to temperature, namely d(EMI FDS)/d(T), may be used as one or more corrective factor(s). It has been determined that a temperature corrective factor is important for improving the accuracy of the determination of the compressive strength of the concrete C.

Other corrective factors may additionally/alternatively used. For example, at step 1850, the humidity measurements from the humidity sensor 140 may be used to determine one or more corrective factors to be used in the subsequent multivariable modelling 1860. Specifically, a derivative of an ultrasound pulse velocity with respect to humidity, namely, d(UPV)/dH), and/or a derivative of the ultrasound pulse frequency domain spectrum with respect to humidity, namely, d(UP FDS)/d(H), and/or a derivative of the electromechanical impedance frequency domain spectrum with respect to humidity, namely d(EMI FDS)/d(H), may be used as one or more corrective factor(s).

At step 1860, multivariable modelling is performed to determine a compressive strength of the concrete C (at step 1870). The multivariable modelling may be performed by the processor 210 of the computing unit 200.

The multivariable modelling is based on the parameters determined at step 1840 together with any corrective factors determined at step 1850. The multivariable model may be a linear regression or a non-linear regression. The multivariable model may be used to determine the compressive strength of the concrete C at a time associated with a set of measurements. The analysis may include a determination of a predicted compressive strength of the concrete C at a future time.

In certain embodiments, the linear regression and/or the non-linear regression may be for the whole dynamic range (e.g. compressive strength of 0 MPa to 60 MPa) or be clustered (e.g. compressive strength of 0 MPa to 10 MPa and 10 MPa to 35 MPa). This is particularly useful as the measured metrics may correlate differently due to the compressive strength range.

In certain embodiments, the multivariable model is based on the ultrasound pulse frequency domain spectrum and, optionally, a specified compressive strength of the concrete C. In certain embodiments, the multivariable model is based on the ultrasound pulse velocity and, optionally, a specified compressive strength of the concrete C. In other embodiments, a specified compressive strength of the concrete C is not used in the multivariable modelling. In such embodiments, no prior information of the concrete C is required for the determination of the compressive strength.

In certain embodiments, the multivariable modelling may include determining the compressive strength of the concrete C by using a neural network model. The neural network model may have been trained using a plurality of concrete samples with known properties. For example, the known properties may include any combination of: the compressive strength, the specified compressive strength, the ultrasound pulse velocity frequency domain spectrum and the electromechanical impedance frequency domain spectrum of the various concrete samples.

The multivariable model may comprise any combination of: linear regression analysis, non-linear regression analysis, a neural network model trained using concrete samples (see above), machine learning (ML), big data (BD), deep learning (DL) and artificial intelligence (AI).

Once the compressive strength of the concrete C is determined for that particular time (e.g. time=0 days), the steps 1830, 1840, 1850, 1860 and 1870 are repeated for different times (e.g. time=1 day). At step 1880, if the compressive strength has been calculated at all required time intervals, the method finishes at step 1890.

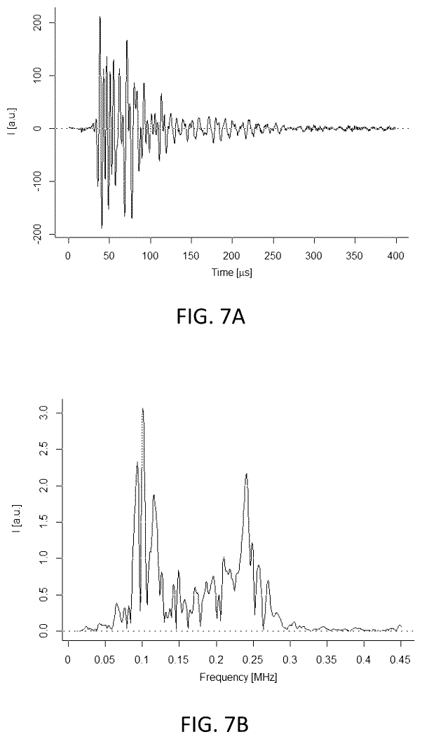

FIGS. 7A and 7B show an exemplary ultrasound pulse measured by the piezoelectric sensor 110. Specifically, FIG. 7A shows an exemplary measurement from the piezoelectric receiver 110b. The graph shows how the intensity of the vibration (measured in arbitrary units a.u.) varies against time. FIG. 7B shows the ultrasound pulse frequency domain spectrum of the signal shown in FIG. 7A. The ultrasound pulse frequency domain spectrum can be obtained by a Fourier Transform (FT)/Fast Fourier Transform (FFT)/wavelet transform of the signal shown in FIG. 7A. The ultrasound pulse response shown in FIG. 7A together with the ultrasound pulse frequency domain spectrum shown in FIG. 7B may be used to determine (at step 1840) any combination of: an ultrasound pulse velocity, a harmonic onset time, a peak frequency, a peak amplitude, a peak area, a peak slope and a peak-to-peak ratio of the ultrasound pulse response.

The ultrasound response is a particularly important metric as it correlates strongly with the compressive strength of the concrete C regardless of the particular mix of the concrete C and doesn't require the sensors to be calibrated prior to use as is typical in the art. Similar data can be obtained for the electromechanical impedance response. Again, the electromechanical impedance response is a particularly important metric as it correlates strongly with the compressive strength of the concrete C regardless of the particular mix of the concrete C and without requiring prior calibration of the sensor.

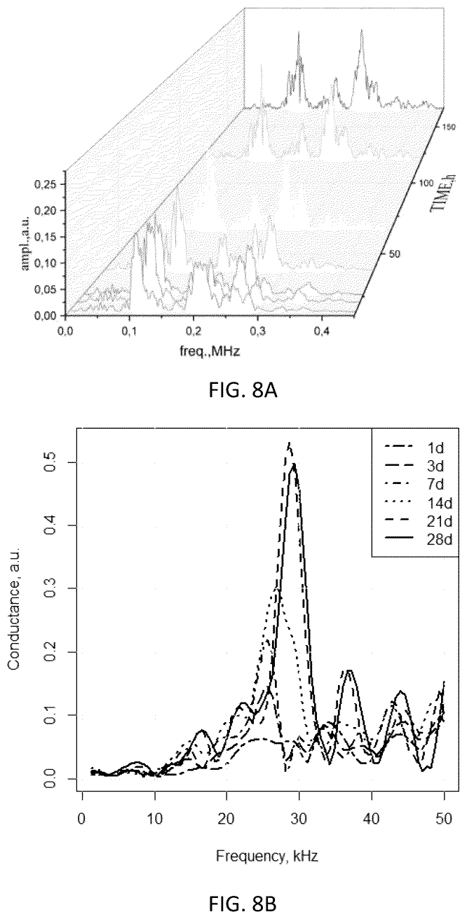

FIG. 8A shows how the ultrasound pulse frequency domain spectrum in concrete C evolves over time. FIG. 8B shows how the electromechanical impedance frequency domain spectrum in concrete C evolves over time. The multivariable model correlates these trends using a linear regression analysis, a non-linear regression analysis, a neural network model trained using concrete samples (see above), machine learning (ML), big data (BD), deep learning (DL), artificial intelligence (AI) and/or any combination thereof to predict the compressive strength based on given parameters. By monitoring these parameters, prior calibration of the sensor is not required and the sensor may function in all types of concrete mix without requiring prior knowledge of the specific concrete mix of concrete C.

While at least one exemplary embodiment has been presented in the foregoing detailed description, it should be appreciated that a vast number of variations exist.

For example, in the above exemplary embodiments, the sensor 100 comprises a number of different sensor elements. However, in alternative embodiments, the sensor 100 may merely comprise a single sensor element, namely the piezoelectric sensor 110. The other sensor elements are purely optional.

Furthermore, the above exemplary embodiments note that the piezoelectric sensor 110 is configured to measure both the ultrasound pulse response and the electromechanical impedance (EMI) response. However, in alternative embodiments, the piezoelectric sensor 110 may be configured to measure the ultrasound pulse response only.

In certain embodiments, a separate sensor may be used to measure the electromechanical impedance response in the concrete C. For example, a second piezoelectric sensor may be used to measure the electromechanical impedance response in the concrete C.

The above exemplary embodiments describe that the piezoelectric sensor 110 comprises both a piezoelectric transmitter 110a and a piezoelectric receiver 110b. However, in alternative embodiments, the piezoelectric sensor 110 may comprise a single piezoelectric transducer that acts as both the transmitter and the receiver (i.e. a transceiver). Optionally, the piezoelectric sensor 110 may further comprise a reflector plate arranged to reflect the ultrasound pulse back to the single piezoelectric transducer.

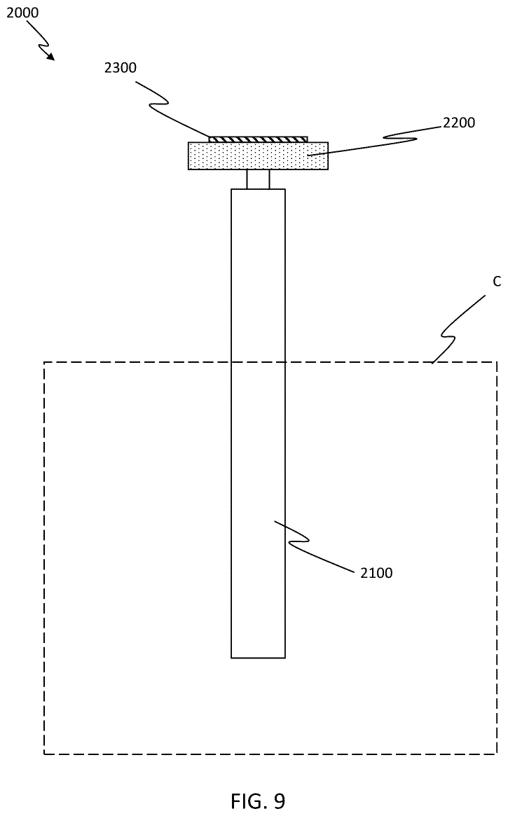

In certain embodiments, the piezoelectric transmitter 110a and/or the piezoelectric receiver 110b (or any piezoelectric transducer described herein) may be in the form shown in FIG. 9. Specially, the piezoelectric transducer 2000 may include a metallic rod 2100 configured to be at least partially embedded in the concrete C, and a bolt 2200 to contact a piezoelectric patch 2300. As shown in FIG. 9, the rod 2100 is placed in concrete C (e.g. after the concrete C has been poured) such that the bolt 2200 may be attached to an exposed end thereof. The piezoelectric transducer 2000 is affixed to the bolt 2200 and is therefore able to transmit/receive vibrations from the bolt 2200, and, in turn, the rod 2100. After use (e.g. after 28 days of measurements), the bolt 2200 together with the piezoelectric transducer 2000 may be removed from the rod 2100 (e.g. by unscrewing) such that the bolt 2200 and piezoelectric transducer 2000 may be reused on other rods 2100. Therefore, the sensor is reusable (e.g. for both ultrasound pulse measurements and electromechanical impedance measurements).

The above exemplary embodiments describe that various parameters are used in the multivariable model. However, in alternative embodiments, only the ultrasound pulse response may be used in the multivariant analysis.

The above exemplary embodiments describe that the multivariable model uses the specified compressive strength of the concrete. However, in alternative embodiments, the multivariable model may additionally/alternatively include any combination of: the specified concrete density, the specified air content and the specified slump. In alternative embodiments, the multivariable model does not use any specified physical parameter of the concrete and instead performs the analysis based on the measured values obtained by sensor 100. In other words, the methods, systems and sensors disclosed herein determine the compressive strength of the concrete C without receiving any prior information of the concrete C.

The above exemplary embodiments describe that the compressive strength of the concrete C is determined. However, in alternative embodiments, a concrete shrinkage may instead be determined. In alternative embodiments, a concrete density and/or a static/dynamic elastic modulus may instead be determined.

The above exemplary embodiments describe that the physical properties of concrete is measured. However, in alternative embodiments, the methods, systems and sensors disclosed herein can be used to measure various physical properties of other materials, such as mortar, grout, gypsum, masonry, asphalt, adhesive, etc.

The methods, systems and sensors disclosed herein can be used to measure compressive strength, shrinkage, density, static/dynamic elastic moduli, glass transition temperature, flexural strength, etc. of any of the materials disclosed herein.

It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing the exemplary embodiment or exemplary embodiments. It should be understood that various changes can be made in the function and arrangement of elements without departing from the scope of the disclosure as set forth in the appended claims and the legal equivalents thereof.

1. A method of determining a compressive strength of concrete, comprising:
(a) measuring, using a piezoelectric sensor, an ultrasound pulse response in the concrete and calculating an ultrasound pulse frequency domain spectrum therefrom;
(b) determining, using a processor and a multivariable model, the compressive strength of the concrete using the ultrasound pulse frequency domain spectrum; and
(c) outputting the compressive strength of the concrete or an indication thereof.

2. The method of claim 1, wherein at least two metrics from the ultrasound pulse response and/or the ultrasound pulse frequency domain spectrum are used in the multivariable model.

3. The method of claim 2, wherein the at least two metrics include two or more of: an ultrasound pulse velocity, a harmonic onset time, a peak frequency, a peak amplitude, a peak area, a peak slope and a peak-to-peak ratio.

4. The method of claim 1, further comprising measuring an electromechanical impedance response in the concrete and calculating an electromechanical impedance frequency domain spectrum therefrom, wherein determining, using the processor and the multivariable model, the compressive strength of the concrete comprises using the ultrasound pulse frequency domain spectrum and the electromechanical impedance frequency domain spectrum.

5. The method of claim 4, wherein the piezoelectric sensor is used to measure the electromechanical impedance frequency response in the concrete.

6. The method of claim 4, wherein at least one metric from the electromechanical impedance frequency domain spectrum is used in the multivariable model.

7. The method of claim 6, wherein at least one metric includes one or more of: a peak frequency, a peak amplitude, a peak area, a peak slope and a peak-to-peak ratio.

8. The method of claim 1, further comprising measuring a temperature in the concrete using a temperature sensor.

9. The method of claim 8, wherein the temperature is used in the multivariable model as a correction factor.

10. The method of claim 1, wherein the piezoelectric sensor is at least partially embedded in the concrete; and/or wherein the piezoelectric sensor is disposed on an external surface of the concrete, optionally, a piezoelectric transducer of the piezoelectric sensor is disposed outside of the concrete.

11. The method of claim 1, wherein the output of the compressive strength of the concrete is used to determine an amount by which the concrete has set.

12. A method of determining a compressive strength of concrete, comprising:
(a) measuring, using a piezoelectric sensor, an ultrasound pulse response in the concrete and calculating, using a processor, an ultrasound pulse frequency domain spectrum therefrom;
(b) measuring, using the piezoelectric sensor, an electromechanical impedance response in the concrete and calculating, using the processor, an electromechanical impedance frequency domain spectrum therefrom;
(c) measuring, using a temperature sensor, a temperature of the concrete;
(d) repeating the measurements of the ultrasound pulse response, the electromechanical impedance response and the temperature over time as the concrete sets, and calculating, using the processor, corresponding ultrasound pulse frequency domain spectrums and electromechanical impedance frequency domain spectrums therefrom; and
(e) determining, using the processor and a multivariable model, the compressive strength of the concrete using the multiple ultrasound pulse frequency domain spectrums and the multiple electromechanical impedance frequency domain spectrums, wherein the multivariable model includes using the temperature as a correction factor; and
(f) outputting a compressive strength of the concrete or an indication thereof.

13. The method of claim 12, wherein: a derivative of an ultrasound pulse velocity with respect to temperature and/or a derivative of the ultrasound pulse frequency domain spectrum with respect to temperature and/or a derivative of the electromechanical impedance frequency domain spectrum with respect to temperature is used as the correction factor.

14. A sensor for determining the compressive strength of concrete, the sensor comprising:
(a) a piezoelectric transmitter; and
(b) a piezoelectric receiver,
(c) wherein the piezoelectric transmitter and the piezoelectric receiver are configured to act together to measure an ultrasound pulse response in the concrete and an electromechanical impedance response in the concrete.

15. The sensor of claim 14, wherein the piezoelectric transmitter and the piezoelectric receiver are spaced apart to define a gap, and wherein the gap is configured to receive a portion of the concrete.

16. The sensor of claim 14, wherein the piezoelectric transmitter and the piezoelectric receiver are implemented in a piezoelectric transducer acting both as the piezoelectric transmitter and receiver.

17. The sensor of claim 14, wherein the sensor is configured to measure the ultrasound pulse response and the electromechanical impedance response with a dwell time of a length to prevent cross talk between the measured ultrasound pulse response and the measured electromechanical impedance response.

18. The sensor of claim 14, wherein sensor is configured to perform multiple measurements of both the ultrasound pulse response and the electromechanical impedance response, and wherein the multiple measurements are at time intervals.

19. The sensor of claim 14, further comprising a temperature sensor configured to measure a temperature of the concrete.

20. The sensor of claim 14, wherein the piezoelectric transmitter and/or the piezoelectric receiver comprises an acoustic coupling layer configured to come into contact with the concrete.
